(19) United States
(12) Patent Application Publication
(10) Pub. No.: US 2004/0047761 A1
(43) Pub. Date: Mar. 11, 2004

(54) Methods and arrangements for disinfection of water lines of a dental unit

(76) Inventors: Pentti Hyvarinen, Helsinki (FI); Arto Huotari, Helsinki (FI)

Correspondence Address: STEINBERG & RASKIN, P.C., 1140 AVENUE OF THE AMERICAS, 15th FLOOR, NEW YORK, NY, 10036-5803, US

(21) Appl. No.: 10/415,019

(22) PCT Filed: Nov. 8, 2001
(86) PCT No.: PCT/FI01/00974

(30) Foreign Application Priority Data
Nov. 8, 2000 (FI) 20002452

(51) Int. Cl. A61L 2/24; A61L 2/16

(52) U.S. Cl. 422/3; 422/28; 422/62; 422/292

(57) ABSTRACT

The invention relates to methods and arrangements for disinfection of water lines of a dental unit, wherein the water lines are filled with a strong chemical, which is allowed to act in the lines for a desired length of time. Firstly, the invention improves the safety of use of such chemicals by realizing the arrangements of the dental unit so as to limit the chances of the user inadvertently admitting the chemical to where it should not be admitted, e.g. into the patient's mouth or into a public water distribution system. Secondly, said arrangement is so implemented as to allow more effective disinfection of the main valve of the water system of the dental unit, the disinfection chemical being supplied via a point upstream of the main valve. The arrangement is so implemented that supply of chemical can not be started before an on-off valve or equivalent placed upstream of the feed point has been closed, and this valve can not be opened before the chemical has been removed from the water lines.

The present invention relates to methods according to claims 1 and 11 and to arrangements according to claims 13 and 21 for disinfection of water lines of a dental treatment apparatus. In particular, the invention relates to solutions in which disinfection is implemented as a separate operation, e.g. at the end of a working day, by supplying a strong disinfectant into the water lines and letting it act in the lines for a longer time, e.g. overnight.

From the perspective of the hygienic requirements concerning dental treatment, one of the most important sub-areas is the microbiological quality of water supplied via dental instruments into the patient's mouth. Preventing microbial growth in the water lines of the dental unit is particularly difficult because, in typical use of the unit, water may stand in the unit at ambient temperature even for long periods, with the result that biofilm, which provides a propitious breeding ground for harmful microbes, tends to be formed on the walls of the narrow water lines.

Countless solutions have been used to fight the problem of microbes in dental units. As for physical disinfection methods, e.g. UV irradiation involves the problem that it only has a local effect, in other words, its effect is almost exclusively limited to the area exposed to irradiation. By using filters, it is in principle possible to prevent microbes from getting into the water lines and into the patient's mouth, but then it is necessary to take care of changing/servicing the filters at sufficiently short intervals, and, on the other hand, the filter itself is a potential source of microbial contamination. Obviously laborious solutions are also those in which water is purified elsewhere before being fed into the dental unit from replaceable supply tanks. On the other hand, even the use of purified water does not completely eliminate the need to periodically wash the water lines and/or change used hoses.

Microbial growth in the water systems of a dental unit can also be prevented by chemical means e.g. by mixing water with a suitable disinfectant in concentrations diluted enough to allow such water still to be supplied to dental instruments, and thus further into patient contact. In these solutions, safety considerations naturally have to be given special attention—an accurate and continuous dosing of the chemical is needed, and strong solution should not get into patient's mouth in any type of malfunction or breakdown. On the other hand, the alternative of using ready-diluted solutions is at least as laborious as is using water purified elsewhere.

The water lines of dental instruments can also be disinfected as a separate operation using strong disinfectants. Some of such substances also have inhibiting effects on the formation of biofilm. When strong solutions are used, it is necessary to make sure that patients will never get into direct contact with the chemical. On the other hand, especially when water to the dental unit is taken from a public tap water network, care should also be taken to ensure that in connection with the disinfection process no chemical may be carried upstream of the main valve of the dental unit and thus eventually further into the public distribution network. Often, authorities and/or water plants have issued technical minimum regulations to exclude such a possibility (e.g. RVV KÄSIKIRJA, RAKENNUSTEN VESIJOHDOT JA VIEMÄRT publication no. 7, Suomen Kunnallistekninen Yhdistys, Helsinki—1979, 6^{th} edition, K. J. Gummerus Oy, Jyväskylä 1979).

Additionally, the main valve forms a special problem in dental units as it by itself forms a potential contamination source. The concentration of disinfectant in the water line portion lying upstream of its point of entry is easily left too low and/or its time of action left shorter there than elsewhere, and especially electrically operated valves may be propitious incubation grounds because of, among other things, their operating temperature.

Patent specifications EP 111 249, WO 95/20366 and U.S. 5,526,841 present arrangements for chemical disinfection of the water lines of dental units, but in all of these, careless or thoughtless action may lead to some of the problems dealt with above. Thus, the object of this invention is to enhance the state of the art expressly from the perspective of safety of the processes of disinfection of the water systems of dental units, as to reduce the possibility of strong disinfection chemicals accidentally getting into the patient's mouth via dental instruments. Further, according to a feature of the invention, its object is to enable better disinfection of the main valve of the water system of the dental unit, yet so that no disinfection solution gets into the dental unit's supply water line—which may be connected to a public water distribution system.

Generally speaking, the object of the invention is to implement the construction and control system of the disinfection system of a dental unit in a manner that will reduce the chances of the user of the dental unit accidentally doing something that would lead to the realization of the above-described risks relating to the use of strong chemicals. These objects will be achieved especially by the solutions defined in the independent claims presented below.

According to a special feature of the invention, the control system of the dental unit comprises control functions that disable the use of at least instruments using water when there is disinfectant in, or when it is being fed into the water system of the unit, in other words, the possibility of disinfectant getting into the patient's mouth via instruments is precluded.

On the other hand, according to a special feature of the invention, the supply container of disinfectant can not be connected to the dental unit if the valve connecting the unit to the supply water line is not closed, and likewise the said valve can not be opened if the disinfectant container is connected to the dental unit—especially when the supply water line is in connection with a public water distribution system.

A further object of the invention is to enable as effective disinfection of the main valve of the dental unit as possible, which is realized by arranging the junction for the supply of disinfecting chemical on the upstream side relative to the main valve, in a way that drifting of the disinfectant upstream from this supply point is prevented.

In the following, the invention and its preferred embodiments will be described in more detail by referring to the attached drawings, wherein

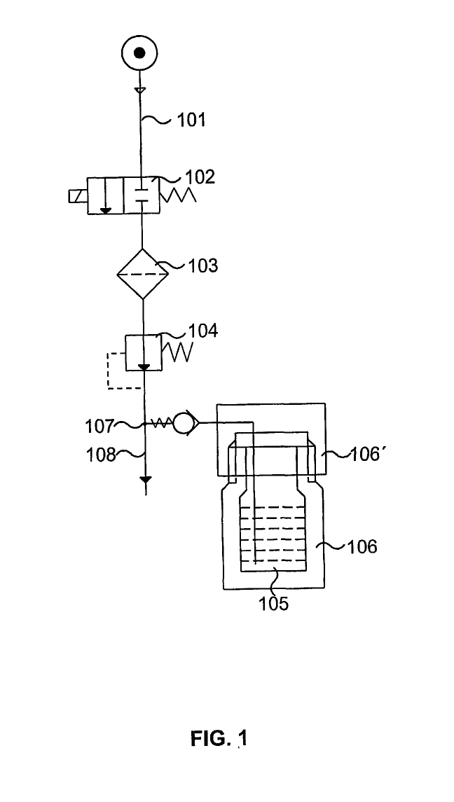

FIG. 1 presents a water line arrangement of a dental unit according to prior art,

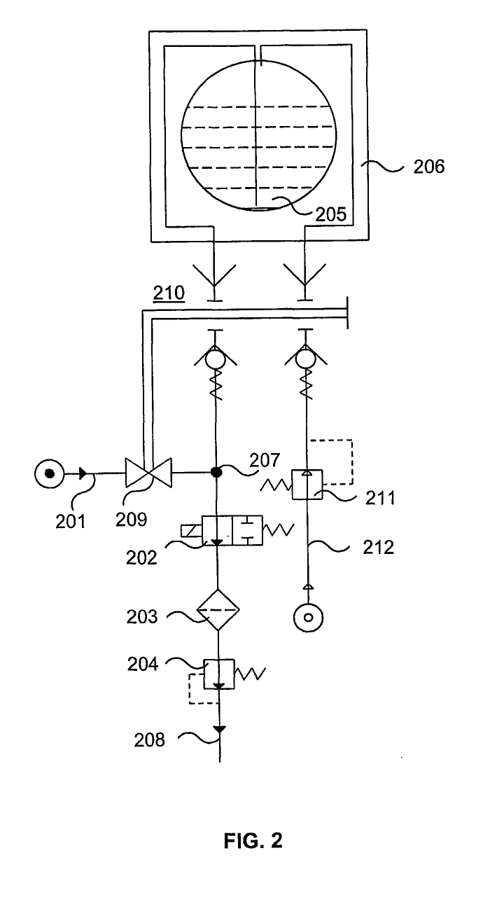

FIG. 2 presents a possible water line arrangement of a dental unit according to the invention, and

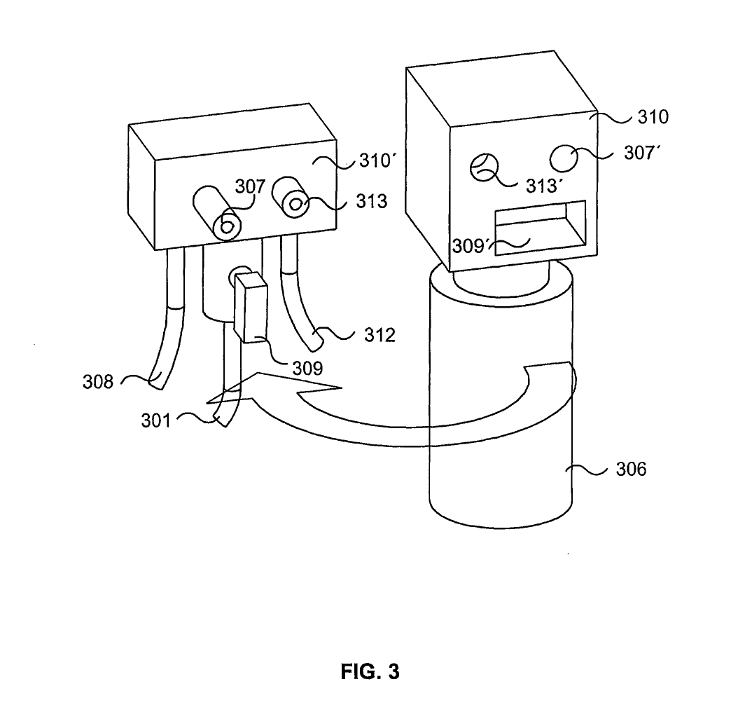

FIG. 3 presents an arrangement according to the invention for connecting a disinfectant container to the water line of a dental unit.

In FIG. 1 there is presented a water line arrangement of a dental unit according to prior art, wherein the main valve 102 of the dental unit is placed in the supply water line 101. Typically, downstream relative to the main valve there is arranged a filtering element 103 and a pressure regulator 104. Using e.g. a compressed air system not shown in FIG. 1, a disinfectant 105 can be driven from its supply container 106, which is connected to a fastening device 106′ being located in the dental unit, via a disinfectant supply junction 107 into the water lines 108 of the dental unit. Typically, for the duration of the disinfecting process, the dental instruments (not shown in FIG. 1) connected to the end of the water lines 108 are placed in a vessel separate from the dental unit, into which the operating-water having been in the water lines 108, as well as the disinfectant 105 after the disinfection time has elapsed, are driven.

In FIG. 2 there is presented a possible dental unit water line arrangement according to the invention, wherein the main valve 202 of the dental unit is placed in the supply water line 201 and downstream of it a filtering element 203 and a pressure regulating element 204. In the solution of the invention, however, the supply junction 207 for disinfectant 205 is so implemented that the container 206 can not be connected to the water line 201, 208 unless connection to the supply water line 201 has been cut off. In the solution according to FIG. 2, according to a preferred embodiment of the invention, this has been realized by providing upstream of the main valve 202 an on-off valve 209 being in a functional contact with coupling elements 210 of the disinfectant container 206 so that connecting the container 206 to the dental unit is only possible if valve 209 is closed. Therefore, for example, if the disinfectant 205 supply container 206 is arranged to be pressurized via a compressed air line 212 provided with a pressure regulator 211, which line may be a branch of the compressed air line of the dental unit, then the disinfectant 205 feed pressure can be arranged to be different, e.g. lower than the operating water pressure in the dental unit, in which case it will be possible to detect, e.g. on the basis of a signal obtained from a pressure gauge (not shown in the figure) placed in the water line 208, that the disinfectant container 206 is connected, and thus to disable, e.g. by means of the control system of the dental unit, the use of dental instruments—at least of those using water. Naturally, the detection of connection of the chemical container—or of the start of supply of the chemical—can also be realized by many other means known as such, e.g. by using a microswitch. In addition, the connected state of the container can be indicated e.g. by a light or a sound signal.

A modern dental unit contains a large number of electric actuators, and consequently the temperature inside its housing may rise locally considerably above the ambient temperature. The check valve or an equivalent element for cutting off the supply water line is therefore advantageously placed outside the dental unit, substantially in the vicinity of its housing or otherwise at a location as far away as possible from any heat emitting elements of the dental unit, to avoid needlessly providing microbes optimal conditions for growth. Naturally, the choice of materials used in said element is also a factor by which its properties as a breeding ground can be affected.

When the supply water line 201 is closed and use of instruments disabled, disinfectant 205 is fed into the water lines 208 of the dental unit. To make sure that the lines 208 are filled completely, it is possible to use e.g. a visually observable colored solution, or to use a quantity of solution known to be sufficient, or the filling up of the lines can be detected by measuring some certain typical quantity of the solution. In the case of long-duration disinfection process, the dental unit is typically shut down at this point—or its control system may comprise an operation sequence “shutting down the unit after the water lines have been filled with disinfectant”.

After the disinfectant 205 has acted for a desired length of time and/or for a length of time defined in the control system, it is driven out from the water lines 208 via the instruments e.g. using compressed air obtainable from the compressed air line 212. This can be done e.g. when the utilization of the dental unit is being started again or automatically after a predetermined time of action. However, as implied above, the use of instruments for dental treatment as well as the connection of water lines 208 to the supply water line 201 are still disabled as long as the disinfectant container 206 remains connected to the water line 208. The detachment of the chemical container 206 can be realized so that the control system of the dental unit controls automatically, or via a compulsory operation sequence, flushing of the water lines 208 after the container 206 has been detached. Thus, the dental unit will only be usable for dental work after the disinfectant 205 has been removed from the water lines 208, the disinfectant container 206 detached, the check valve 209 opened and the water lines 208 flushed. The arrangement preferably also comprises appropriate means for detecting the execution of the flushing operation, so that the user can not accidentally omit the flushing of the lines, either.

The flushing of the water line can also be arranged to be realized by supplying e.g. flushing water purified elsewhere into the line 208 via the same junction 207 through which the disinfectant was supplied. Different alternatives for implementing this solution in practice are obvious to a man skilled in the art—e.g. by using a separate replaceable purified water container attachable to the coupling elements 210 or by providing a possibility to connect two containers to this coupling arrangement, yet taking account of the requirements of operational safety according to the invention. This manner of implementation would naturally involve the loss of some of the convenience and ease of use provided by other embodiments of the invention.

In FIG. 3 there is presented an arrangement according to the invention for connecting the disinfectant container to a water line of a dental unit. Placed in the supply water line 301 at a location upstream of the main valve (not shown in the figure) and the disinfectant supply junction 307 is an on-off valve 309, which is always open when the dental unit is being used for dental work, permitting flow of water toward the water lines 308 of the dental unit. The coupling element 310 of the disinfectant container 306 is so implemented that the container 306 can not be coupled to corresponding elements 310′ (309) in the dental unit unless the check valve 309 is closed. The container 306 connected to the dental unit can be pressurized e.g. in a manner corresponding to that presented in FIG. 2 by using compressed air obtained via a compressed air line 312 and its supply junction 313, and the disinfection chemical can be fed into the water lines 308 of the dental unit via its supply junction 307. As long as the disinfectant container 306 remains connected to the dental unit, the on-off valve 309 can not be opened.

Naturally, functional connection of the on-off valve 309 to the coupling elements 310, 310′ of the disinfectant container 306/dental unit can be realized in many different ways, e.g. by using such a quick-coupling that, when engaging or disengaging, it at the same time closes/opens the on-off valve 309 or an equivalent element.

According to a preferred embodiment of the invention, the connecting pipe of the disinfectant supply junction 307 is made longer than the corresponding pipe of the compressed air supply junction 313 to ensure that, when the chemical container 306 is being detached, a possible pressure difference between, it and the environment will be equalized before the chemical supply junctions 307, 3071 are released from the coupling. In this way, the splashing of any chemical residues from the disinfectant container in connection with its release can be largely prevented.

In the foregoing, the invention has largely been described by only referring to embodiments as illustrated in the attached FIGS. 2 and 3, in which, however, many details may be varied within the framework of the invention within the scope defined in the following claims. Let it be stated, for example, that although in the above description reference has mainly been made to the instrument water lines of the dental unit, the invention can naturally be advantageously applied to the entire water system of the dental unit. On the other hand, although in the invention it is naturally possible to use any suitable disinfection chemical, it is preferable to use a substance that, besides disinfection, also contributes towards removing a possible biofilm layer from the walls of the water lines. Moreover, as already stated in places above, many steps in the disinfection process can, if so desired, be partially or completely automated and/or realized in compulsory phases.

1. Method for disinfection of water lines of a dental unit, in which method the use of instruments is interrupted for the duration of the disinfection process, the supply of water into said lines is cut off and the lines are filled with disinfectant, characterized in that, starting at the latest from the instant when the supply of disinfectant into the water lines of the dental unit is started and lasting at least as long as said lines contain disinfectant, the dental unit is so controlled as to disable the use of at least dental instruments using water and so as to keep the water supply connection to said water lines closed, said control functions being realized by control means of the dental unit and/or by its other, such as mechanical structures.

2. Disinfection method according to claim 1, characterized in that at least some of said control functions are activated on the basis of a detection signal obtained from means provided in the dental unit to detect the coupling of a disinfectant container to the dental unit and/or from means detecting the start of supply of disinfectant.

3. Disinfection method according to any of the claims 1 or 2, characterized in that said water supply connection is cut off by a mechanically compulsory-phased action before said container is connected to its coupling element, or that said connection is cut off automatically when the container is connected to its coupling element provided in the dental unit.

4. Disinfection method according to any of the claims 1-3, characterized in that the disinfectant is fed into the water lines by using compressed air obtained from the compressed air line of the dental unit, e.g. at a pressure lower than the operating pressure in the water lines of the dental unit.

5. Disinfection method according to claim 4, characterized in that said control functions, at least as regards disabling the use of instruments, are activated on the basis of a detection signal obtained from a pressure sensor measuring the pressure of the operating-water in the dental unit.

6. Disinfection method acoording to any of the claims 1-5, characterized in that the disinfectant is allowed to act in the water lines for a desired length of time and/or for a time predefined in the control system, where it is removed from said lines, e.g. by using compressed air obtained from the compressed air line of the dental unit.

7. Disinfection method according to any of the claims 1-6, characterized in that said control function are terminated after the disinfectant container he been release from its coupling eldest and, at least as regards disabling the use of instruments, preferably only after the water lines have been flushed.

8. Disinfection method according to claim 7, characterized in that said flushing is performed as an automatic or as a compulsory-phased sequence of “release of disinfectant container, opening of water supply junction and flushing of water lines”.

9. Disinfection method according to any of the claims 7 or 8, characterized in that the disablement of the application of instruments is ended based an a signal received upon completion of flushing of the water lines.

10. Disinfection method according to any of the claims 1-9, characterized in that said ply water used in the dental unit is obtained from a public water distribution system.

11. Method for disinfection of water lines of a dental unit, in which method the use of instruments is untempted for the duration of the disinfection process, the supply of water into said lines is cut off and the lines are filled with disinfectant, characterized in that the disinfectant is fed into said water line upstream of the main valve of the dental unit, and cutting off of the water supply connection to said water win for the duration of the disinfection process is realized by using means for cutting off the supply connection placed upstream of said disinfectant feed point.

12. Disinfection method according to any of the claims 1-9, or according to the characterizing part of any one of them, and/or according to claim 10, characterized in that the disinfectant used is preferably a substance that also contributes towards releasing biofilm formed on the surfaces of the water lines.

13. Arrangement for disinfection of water lines of a dental unit, comprising means for cutting off the supply of water to said water lines and means for filling said water lines with a disinfectant, characterized in that the arrangement comprises a control system of the dental unit and/or other, such as mechanical means, which disable the functions
supply of water into the waterlines and
use of at least dental instruments using water, starting at the latest at an instant when the supply of disinfectant into the water lines of the dental unit is started and lasting at least as long as said lines contain disinfectant.

14. Disinfection arrangement according to claim 13, characterized in that the dental unit is provided with means for detecting the connection of a disinfectant container to the dental unit and/or the start of the supply of disinfectant, and means for activating at least some of said control functions on the basis of this detection.

15. Disinfection arrangement according to any of the claims 13 or 14, characterized in that the means for connecting the disinfectant container to the dental unit are functionally connected to the means for cutting off the water supply connection so that the said container can not be connected to its coupling element arranged in the dental unit unless the water supply connection has been cut off, or that connecting said container to the dental unit automatically cuts off said supply connection.

16. Disinfection arrangement according to any of the claims 13-15, characterized in that the supply of disinfectant from the disinfectant container into the water lines and/or its removal from said lines has been arranged to be done by using compressed air obtained from the compressed air line of the dental unit, said arrangement comprising e.g. a branch from said compressed air line to the disinfectant container, the compressed air line leading to said branch being provided with a pressure regulating element for producing a disinfectant feed pressure that is different, e.g. lower, than the operating pressure of water in the dental unit.

17. Disinfection arrangement according to claim 16, characterized in that the control system of the dental unit comprises means for detecting a predetermined change in the signal obtained from a pressure gauge placed in the water line of the dental unit, and means for activating on the basis of detection of this change at least some of said control functions, such as especially the disablement of the use of dental instruments.

18. Disinfection arrangement according to any of the claims 13-17, characterized in that the means for connecting the disinfectant container to the dental unit are functionally connected to the means for cutting off the water supply connection so that the water supply connection can not be opened as long as said container remains coupled to its connector arranged in the dental unit, and/or that detaching said container from the dental unit automatically opens said supply connection.

19. Disinfection arrangement according to any of the claims 13-18, characterized in that the structure for coupling the disinfectant container to the dental unit comprises two pipe supplies of different lengths, of which the pipe for compressed air is shorter than the pipe for disinfectant.

20. Disinfection arrangement according to any of the claims 13-19, characterized in that the supply water for the dental unit has been arranged to be taken from a public water distribution system.

21. Arrangement for disinfection of water lines of a dental unit, comprising means, such as main valve, for cutting off the supply of water into said water lines and means for filling them with a disinfectant, characterized in that the junction for the supply of disinfectant is placed upstream of said main valve, and that the arrangement comprises other means, placed upstream of said supply junction, for cutting off the water supply connection.

22. Disinfection arrangement according to any of the claims 13-20, or according to characterizing part of any one of them, and/or according to claim 21, characterized in that it is preferably in connection with the entire water system of the dental unit.
